(19) United States
(12) Patent Application Publication
(10) Pub. No.: US 2009/0215085 A1
(43) Pub. Date: Aug. 27, 2009

(54) Proteomic Fingerprinting of Human IVF-Derived Embryos: Identification of Biomarkers of Developmental Potential

(75) Inventors: Sergio C. Oehninger, Norfolk, VA (US); O. John Semmes, Newport News, VA (US); Lisa H. Cazares, Chesapeake, VA (US); Silvina Bocca, Norfolk, VA (US)

Correspondence Address: WILMERHALE/DC, 1875 PENNSYLVANIA AVE., NW, WASHINGTON, DC, 20006, US

(73) Assignee: Eastern Virginia Medical School, Norfolk, VA (US)

(21) Appl. No.: 12/162,471

(22) PCT Filed: Jan. 29, 2007
(86) PCT No.: PCT/US07/02456

§ 371 Date: Feb. 12, 2009

Related U.S. Application Data
(60) Provisional application No. 60/762,492, filed on Jan. 27, 2006.

(51) Int. Cl. G01N 33/53; C12Q 1/02; C12M 1/00; B01D 59/44

(52) U.S. Cl. 435/7.21; 435/29; 435/287.2; 250/282

(57) ABSTRACT

The present invention discloses biomarkers and biomarker combinations that have prognostic value as predictors of the developmental potential of individual IVF-derived human embryos. In particular, the biomarkers of this invention are useful to classify an embryo with implantation competence after uterine transfer or implantation incompetence. In addition, the biomarkers can be detected by non-invasive methods that do not harm the developing embryo. Also disclosed are kits for the prediction of developmental potential that detect the biomarkers of the invention, as well as methods using a plurality of classifiers to make a probable diagnosis of developmental potential.

### BACKGROUND OF THE INVENTION

Up to one in six couples suffers from infertility. For many, in vitro fertilization (IVF) therapies are the only treatment of choice. Although this therapy has helped hundreds of thousands of couples to have children, success rates remain relatively low, with a live birth rate per treatment cycle of around 35% worldwide. Those IVF therapies that lead to an initial successful pregnancy test result are too often followed a few weeks later by an ultrasound that presents a new obstacle—multiple pregnancies.

While a single pregnancy and live birth is the ultimate goal of patients and physicians alike, multiple pregnancies, unfortunately, are one of the leading causes of morbidity related to infertility therapy and also result in a major economic burden for the health care system. While the risks of conceiving multiple gestations are high when employing assisted reproductive technologies, only a few centers offer selective reduction (reduction of the number of fetuses within a multiple pregnancy) as an option to couples. The expectant mothers who attempt to carry multiple gestations to term face an increased risk for developing complications of pregnancy such as gestational diabetes, pre-eclampsia (toxemia), pregnancy-induced high blood pressure, preterm labor, vaginal/uterine hemorrhage, and other complications. The risks to the fetuses and children are mostly related to premature delivery and can result in very severe complications. Compared to a singleton, a twin is about five times more likely to die in the first year of life. For a triplet, this risk is about 13 times that of a singleton. The risk of having a lifelong handicap (e.g., cerebral palsy, mental retardation) is increased about 10 times for twins compared to singles, and these risks are substantially higher for triplets. Quadruplet and other high-order pregnancies are much riskier. Fortunately, in accordance with current embryo transfer policies, clinics are moving towards transferring fewer embryos and pregnancies beyond triplets are becoming increasingly rare with IVF.

There is general agreement that a solution to these problems would be to transfer only one (in patients with the most favorable prognosis) or two embryos (in patients with below average prognosis) following IVF. Various prognosis groups are determined by factors such as the patient's age, whether embryos are available for freezing, and the patient's history of IVF failures. Although the ultimate objective of transferring a single embryo in all cases will likely not be attained in the immediate future, infertility specialists continue to strive for the most appropriate number of embryos to transfer to have the ideal balance between pregnancy versus no pregnancy and single pregnancy versus multiple pregnancies.

In past years, the majority of embryo transfers was performed on day 3 (after egg retrieval and subsequent in vitro fertilization) at the “cleavage stage” when the embryos have four to eight cells. One problem with this is that day 3 embryos are normally found in the fallopian tubes, not in the uterus. The implantation process begins about 3 days later, after blastocyst formation (roughly 100 cell stage embryo). A healthy blastocyst should begin hatching from its outer shell, called the zona pellucida, by the end of the sixth day. Within about 24 hours after hatching, it should begin to implant into the lining of the mother's uterus. The other problem with transferring on day 3 is that many embryos at that stage do not have the capacity to continue development and become high-quality blastocysts. Recent developments have facilitated the formulation of stage-specific embryo culture media designed to extend the in vitro culture system process and support embryo development throughout the pre-implantation period, but only 25% to 60% of day 3 embryos reach the blastocyst stage. By choosing the principal blastocysts on day 5, however, one can allow nature to further differentiate the embryos with the best developmental potential i.e. viable embryos with the highest likelihood of survival, implanting successfully after uterine transfer and leading to a full term pregnancy. Resultant implantation rates attained with the culture and transfer of human blastocysts are indeed higher than those associated with the transfer of cleavage stage embryos to the uterus. The only caveat is that there are remarkable variations in a patient's ability to produce blastocysts and there are currently no reliable methods to determine which of the day 3 embryos will be viable and robust on day 5 (Day 3 morphology is a poor predictor of blastocyst quality in extended culture. 2000. Graham et al., Fertil Steril. 74(3):495-7). Thus, a risk of attempting blastocyst transfer is the possibility that no embryos will be available for transfer on day 5. Therefore, in these latter cases, the tendency has been to transfer more embryos on day 3 in an attempt to achieve good implantation rates.

It is the state of the art to select human IVF-derived embryos for uterine transfer based on microscopic observations during the pre-implantation ex vivo culture period. Such features include nucleoli apposition at the pronuclear stage, time of embryo first division (cleavage) and embryo cell morphology scoring and cleavage stage at transfer (typically performed on day 2-5 of culture). The predictive value of these observations for each embryo's developmental potential is very limited. There appears to be a high incidence of early pregnancy loss after in vitro fertilization with a biochemical pregnancy rate of 18% and a spontaneous abortion rate of 27% (Early pregnancy loss in in vitro fertilization (IVF) is a positive predictor of subsequent IVF success. 2002. Bates and Ginsburg. Fertil Steril. 77(2):337-41). Thus, although there have been improvements in IVF techniques, implantation failure may be the cause for a large number of losses with embryo transfer and this implantation competence aspect of developmental potential is one of the areas that needs to be optimized. To overcome the limitations above, multiple embryos are typically transferred in the clinical setting, which may result in higher pregnancy rates, but may also lead to the major complication of multiple gestations as discussed above.

Blastocyst transfer currently offers the best balance between achieving higher full term pregnancy rates while minimizing the risk of multiple pregnancy, but significant concern remains that a mere reduction in the number of day 5 embryos transferred may still appreciably lower the overall chances of pregnancy for many couples. The goal of transferring a single embryo and having every treatment cycle result in a single full term pregnancy and live birth for every couple could only be achieved if embryos with the highest developmental potential could be chosen.

These limitations fuel the ongoing search to identify biomarkers that correlate with survival, implantation competence and subsequent full term pregnancy resulting from IVF therapies. Interleukin-(IL) 18 has been investigated for its use as a prognostic factor in inadequate uterine receptivity (Detectable levels of interleukin-18 in uterine luminal secretions at oocyte retrieval predict failure of the embryo transfer. 2004. Ledee-Bataille et al., Hum Reprod. 2004. 19(9):1968-73). The level of matrix metalloproteinase-9 (MMP-9) in pre-ovulatory follicular fluid has been investigated for its use as a pre-diagnosis marker for successful implantation in human IVF (The expression of matrix metalloproteinase-9 in human follicular fluid is associated with in vitro fertilization pregnancy. 2005. Lee et al., BJOG. 112(7):946-51). In U.S. Pat. No. 5,635,366, the level of 11β-hydroxysteroid dehydrogenase (11β-HSD) in a biological sample from a female patient has been shown to determine the probability of establishing pregnancy in said subject by IVF. U.S. Pat. No. 6,660,531 discloses a method of determining the probability of an in vitro fertilization (IVF) or embryo transfer (ET) being successful by measuring the levels of relaxin, directly in the serum or indirectly by culturing granulosa lutein cells extracted from the patient, as part of an IVF/ET procedure. These non-invasive methods may predict inadequate uterine receptivity and help lead to a more accurate overall prognosis for each prospective mother, but they are independent of embryo quality and each cohort of embryos can represent a heterogeneous population containing both normal and degenerative eggs with varying degrees of developmental potential, chromosomal abnormalities, morphology characteristics, etc.

One study suggests that analyzing the regulation of apoptosis in the pre-implantation embryo may be predictive of both embryo viability and developmental potential, but these methods are invasive and may harm the embryo (Apoptosis in mammalian pre-implantation embryos: regulation by survival factors. 2000. Brison. Hum Fertil (Camb). 3(1):36-47).

There are reports of non-invasive methods that detect individual proteins in the culture medium of developing human embryos, which show low predictive value for implantation competence (hCG; Analysis of chorionic gonadotrophin secreted by cultured human blastocysts. 1997. Lopata et al., Mol Hum Reprod. 3(6):517-21, soluble HLA antigen; Expression of sHLA-G in supernatants of individually cultured 46-h embryos: a potentially valuable indicator of ‘embryo competency’ and IVF outcome. 2004. Sher et al., Reprod Biomed Online. 9(1):74-8). In addition, there are publications related to the selection of human IVF embryos by studying carbohydrate metabolism and amino acid turnover during in vitro culture (Identification of viable embryos in IVF by non-invasive measurement of amino acid turnover. 2004. Brison et al., Hum. Report. 19(10):2319-24 and Non-invasive assessment of human embryo nutrient consumption as a measure of developmental potential. 2001. Gardner et al., Fertil. Steril. 76(6):1175-80). Similarly, more recent studies have looked at the pattern of depletion and appearance of a mixture of amino acids by other IVF-derived mammalian embryos (Amino acid metabolism of the porcine blastocyst. 2005. Humpherson et al., Theriogenology. 64(8):1852-66).

The effectiveness of any diagnostic test depends on its specificity and selectivity, or the relative ratio of true positive, true negative, false positive and false negative diagnoses. Methods of increasing the percent of true positive and true negative diagnoses for any condition are desirable medical goals. Overall, despite the identification of individual biomarkers that may aid in predicting the developmental potential of an embryo, none have yet emerged that has changed current IVF practices. Given the complexity of the genetic and molecular alterations that occur in each developing embryo, the expression patterns reflecting these complex changes, in addition to individual molecular changes themselves, may also hold vital information in diagnosing the developmental potential of an embryo.

Proteomic research, which looks at the expression profile of multiple proteins within a complex sample, therefore, has promising clinical applications. The primary aim of clinical proteomics is to identify differentially expressed biomarkers, by comparing the proteomic profiles of differing physiological states, which can be used for diagnosis and therapeutic intervention. In addition to immunoassays, proteomic research has traditionally involved two-dimensional gel electrophoresis to detect protein expression differences in body fluid specimens between groups (Srinivas, P. R., et al., Clin Chem. 47:1901-1911 (2001); Adam, B. L., et al., Proteomics 1:1264-1270 (2001)). Although two-dimensional polyacrylamide gel electrophoresis (2D-PAGE) has been the classical approach in exploring the proteome for separation and detection of differences in protein expression, it has its limitations in that it is cumbersome, labor intensive, suffers reproducibility problems, and is not easily applied in the clinical setting.

One of the recent technological advances in facilitating protein profiling of complex biologic mixtures is mass spectrometry (MS). Mass spectrometry-based proteomics have made it possible to detect and quantitate individual proteins and multiple proteins simultaneously while analyzing the entire proteome of biological samples. The matrix-assisted laser desorption/ionization (MALDI)-time of flight (TOF)-MS method, where protein solutions are typically premixed with a matrix and dried on a passive surface, can provide direct identification of each individual protein present in a complex biological sample. After characterizing the protein peaks in a biological sample, the sample can be further analyzed to generate a protein profile or protein signature.

Similarly, surface-enhanced laser desorption/ionization time of flight mass spectrometry (SELDI-TOF-MS) detects proteins bound to a protein chip array and facilitates the identification of a signature protein profile (Kuwata, H., et al., Biochem. Biophys. Res. Commun. 245:764-773 (1998); Merchant, M. et al., Electrophoresis 21:1164-1177 (2000)). MALDI and SELDI technology have numerous advantages over 2D-PAGE: they are much faster, have a high throughput capability, require orders of magnitude lower amounts of the protein sample, can effectively resolve low and higher mass proteins (500-100,000 Da), and are directly applicable for assay development.

Applications of mass spectrometry-based quantitative proteomics show great potential for the early detection of prostate, breast, ovarian, bladder, and head and neck cancers (Li, J., et al., Clin. Chem. 48:1296-1304 (2002); Adam, B., et al., Cancer Res. 62:3609-3614 (2002); Cazares, L. H., et al., Clin. Cancer Res. 8:2541-2552 (2002); Petricoin, E. F., et al., Lancet 359:572-577 (2002); Petricoin, E. F. et al., J. Natl. Cancer Inst. 94:1576-1578 (2002); Vlahou, A., et al., Amer. J. Pathology 158:1491-1502 (2001); Wadsworth, J. T., et al., Arch. Otolaryngol. Head Neck Surg. 130:98-104 (2004)).

SELDI proteomic technology has been used to extract clinically relevant biomarkers of intra-amniotic inflammation. Patients with intra-amniotic inflammation that deliver preterm have a distinctive amniotic fluid proteomic profile and this methodology may identify the subgroup of patients that might benefit most from interventions to prevent fetal damage in utero (Proteomic biomarker analysis of amniotic fluid for identification of intra-amniotic inflammation. 2005. Buhimschi et al., BJOG. 112(2):173-81). In addition, several biomarkers were identified in female serum samples that may discriminate an ectopic pregnancy from an intrauterine pregnancy (A serum proteomics approach to the diagnosis of ectopic pregnancy. 2004. Gerton G L, Fan X J, Chittams J, Sammel M, Hummel A, Strauss J F, Barnhart K. Ann N Y Acad Sci. 1022:306-16). These studies demonstrate the potential of the SELDI platform for reproducible and consistent analysis of human samples and show that this approach could be employed for the analysis of other biofluids or test samples.

However, to date, as far as the inventors are aware, the use of MALDI or SELDI has not been employed to identify a unique biomarker or a protein fingerprint of developmentally competent IVF-derived human embryos themselves or the media in which they are cultured ex vivo.

Identifying biomarkers and/or protein profiles, during the days of in vitro culture, that will allow for the selection of embryos with the highest developmental potential, will complement existing methods to select embryos for transfer, which are currently based solely on microscopic assessments. Clinical proteomics will allow physicians specializing in IVF to efficiently and rapidly select a single embryo from a given cohort of growing embryos, ensuring that the embryo with the most optimal developmental potential can be transferred. Moreover, proteomics may help select embryos earlier than it is presently possible with existing microscopic assessments and lead to minimizing, or ultimately avoiding, the current extended culture period in vitro. Furthermore, the identification of specific predictive biomarkers within a diagnostic protein profile or algorithm can be used to develop more rapid and cost-effective methods such as ELISA, antibody-capture, microchips or kits which can be employed by the average medical technician who may not be trained in MALDI or SELDI technology. Such a clinical application of proteomics will thus have a dramatic impact in assisted reproductive technologies as it will not only eliminate the incidence of multiple gestations but will also increase the likelihood of each IVF cycle resulting in a full term pregnancy. Moreover, the lack of insurance coverage in most cases, compounded by the emotional investment of infertile couples, makes repeat cycles after IVF failures undesirable while multiple pregnancies represent a major economic burden for the health system. The present invention addresses these and other important ends.

### BRIEF SUMMARY OF THE INVENTION

The present invention is directed to biomarkers that have prognostic value as predictors of the developmental potential of human IVF-derived embryos. These biomarkers are differentially present in the culture medium used to support the ex vivo growth of IVF-derived human embryos that have developmental potential and in the culture medium used to support the ex vivo growth of IVF-derived human embryos that do not have developmental potential. The present invention also provides sensitive methods and kits that can be used as an aid in the diagnosis of the developmental potential of IVF-derived human embryos by detecting these novel markers. The detection and measurement of these biomarkers, alone or in combination, in culture medium samples, provides information that can be correlated with a prognosis of the developmental potential of IVF-derived human embryos. All the biomarkers are characterized by molecular weight. The biomarkers can be resolved from other proteins in a sample by, e.g., chromatographic separation coupled with mass spectrometry, or by traditional immunoassays. In preferred embodiments, the method of resolution involves Matrix-Assisted Laser Desorption/Ionization (MALDI) mass spectrometry or Surface-Enhanced Laser Desorption/Ionization (SELDI) mass spectrometry.

In one form of the invention, a method for aiding in, or otherwise making, a diagnosis of developmental potential includes detecting at least one biomarker in a test sample from the IVF-derived embryo culture medium.

The biomarkers have a molecular weight selected from the group consisting of about 2454±5, 2648±5.3, 2665±5.3, 2684±5.4, 2778±5.6, 4093±8.2, 37820±76, 45873±92, 282390±565 and 435170±870 Daltons. The method further includes correlating the detection with a prognostic prediction of developmental potential.

The method further includes correlating the detection and measurement of at least one biomarker with a prognostic prediction of a successful implantation and subsequent full term pregnancy end point after uterine transfer of fresh or frozen embryos. In another form of the invention, the method will facilitate making a prognostic prediction of which cryopreserved embryos will have the highest likelihood of survival (post-thaw), followed by the highest implantation competence after transfer, followed by the highest likelihood of producing a full term pregnancy.

In one embodiment, the correlation takes into account the amount of the marker or markers in the sample and/or the frequency of detection of the same marker or markers in a characterized control.

In another embodiment, gas phase ion spectrometry is used for detecting the marker or markers. For example, laser desorption/ionization mass spectrometry may be used.

In one embodiment, matrix-assisted laser desorption/ionization (MALDI) may be used to provide direct identification of each individual biomarker present in the biological sample. After characterizing the protein peaks in the biological sample, the sample may be further analyzed to generate a protein profile or protein signature.

In another embodiment, laser desorption/ionization mass spectrometry used to detect markers comprises: (a) providing a substrate comprising an adsorbent attached thereto; (b) contacting the sample with the adsorbent; and (c) desorbing and ionizing the marker or markers with the mass spectrometer. Any suitable adsorbent may be used to bind one or more markers. For example, the adsorbent on the substrate may be a cationic adsorbent, an antibody adsorbent, etc.

In another embodiment, an immunoassay can be used for detecting the marker or markers.

In another embodiment, NMR spectrometry may be used for detecting a biomarker or biomarkers within the sample.

In accordance with the present invention, at least one biomarker may be detected. It is to be understood, and is described herein, that one or more biomarkers may be detected and subsequently analyzed, including several or all of the biomarkers identified. Further, it is to be understood that the failure to detect one or more of the biomarkers of the invention, or the detection thereof at levels or quantities that may correlate with implantation competence, may be useful and desirable as a means of selecting the most favorable embryos to transfer, and that the same forms a contemplated aspect of the present invention.

In yet another aspect of the invention, methods of using a plurality of classifiers to make a probable prediction of implantation competence are provided. In one form of the invention, a method includes a) obtaining mass spectra from a plurality of samples from successful implantations and subsequent live births, successful implantations that do not result in live births and failed implantation samples; b) applying a decision tree analysis to at least a portion of the mass spectra to obtain a plurality of weighted base classifiers comprising a peak intensity value and an associated threshold value; and c) making a prognostic prediction of implantation competence and full term pregnancy based on a linear combination of the plurality of weighted base classifiers. In certain forms of the invention, the method may include using the peak intensity value and the associated threshold value in linear combination to make a probable prediction or diagnosis.

It is a further object of the invention to provide computer program media storing computer instructions therein for instructing a computer to perform a computer-implemented process for developing and/or using a plurality of classifiers to make a prognosis of developmental potential using at least one biomarker having a molecular weight selected from the group consisting of about 2454±5, 2648±5.3, 2665±5.3, 2684±5.4, 2778±5.6, 4093±8.2, 37820±76, 45873±92, 282390±565 and 435170±870 Daltons.

In another aspect of the invention, kits are provided and may be utilized in the detection of the biomarkers described herein and may otherwise be used to diagnose, or otherwise aid in the diagnosis of developmental potential. In one embodiment, the kit may employ MALDI to detect individual biomarkers present in the biological sample. In one form of the invention, a kit may include an adsorbent comprising at least one capture reagent thereto, wherein the capture reagent is capable of retaining at least one biomarker having a molecular weight selected from the group consisting of about 2454±5, 2648±5.3, 2665±5.3, 2684±5.4, 2778±5.6, 4093±8.2, 37820±76, 45873±92, 282390±565 and 435170∓870 Daltons; and instructions to detect the biomarker by contacting a test sample with the adsorbent and detecting the biomarker retained by the adsorbent. In one form of the invention, the adsorbent may be a SELDI probe, an antibody that specifically binds a biomarker, a cation exchange chromatography adsorbent, an anion exchange chromatography adsorbent or a biospecific adsorbent.

In yet another embodiment of the invention, the kit may include instructions on how to use the adsorbent to detect a plurality of biomarkers.

### BRIEF DESCRIPTION OF THE FIGURES

FIG. 1 depicts an average MALDI spectra performed on day 2 embryo culture medium samples via WCX magnetic bead fractionation exhibiting the most discriminatory peaks at about 2454, 2648, 2665, 2684 and 2778 Daltons, which were underexpressed in the media samples from IVF-derived embryos which produced a positive implantation. The spectra represents peptides or proteins present in the biological sample, either in the embryo culture medium itself, those taken up by the embryo or those secreted from the embryo; the darker line represents media samples from successful implantations of transferred IVF-derived embryos; the lighter shaded line represents media samples from failed implantations of transferred IVF-derived embryos; the x-axis is mass per charge (m/z); the Y-axis is relative intensity.

FIG. 2 depicts a scatter plot of the distribution of peak intensities for m/z 2454 between the two sample groups of day 3 embryo media. Seventy percent of the media samples from embryos on Day 3, producing a pregnancy, are below the average for the media samples not producing a pregnancy for this peak.

FIG. 3 depicts a scatter plot of the distribution of peak intensities for m/z 2648 between the two sample groups of day 3 embryo media. Eighty eight percent of the media samples from embryos on Day 3, producing a pregnancy, are below the average for the media samples not producing a pregnancy for this peak.

FIG. 4 depicts a scatter plot of the distribution of peak intensities for m/z 2684 between the two sample groups of day 3 embryo media. Sixty eight percent of the media samples from embryos on Day 3, producing a pregnancy, are below the average for the media samples not producing a pregnancy for this peak.

FIG. 5 depicts a scatter plot of the distribution of peak intensities for m/z 2778 between the two sample groups of day 3 embryo medias. Seventy three percent of the media samples from embryos on Day 3, producing a pregnancy, are below the average for the media samples not producing a pregnancy for this peak.

FIG. 6 depicts results of a MALDI MS/MS LIFT spectra MASCOT search identifying the peak at m/z 2454 as a fragment of Opiomelanocortin. The top panel is the probability MOWSE score of 76 where scores greater than 67 are significant. The bottom panel shows the peptide identified in the context of the entire protein sequence.

FIG. 7 depicts MALDI MS/MS Lift spectra of the three peaks at m/z 2684, 2666, and 2684. The fragmentation pattern is identical, indicating that these peaks are attributed to the same peptide sequence.

FIG. 8 depicts results of a MALDI MS/MS LIFT spectra MASCOT search identifying the peak at m/z 2665.6 as a fragment of Apolipoprotein A1. The top panel is the probability MOWSE score of 93 where scores greater than 64 are significant. Below is the peptide sequence identified.

FIG. 9 depicts the peptide sequence of the Apolipoprotein A1 fragment identified in the context of the entire protein sequence.

FIG. 10 depicts the fragmentation spectra of the Apolipoprotein A1 peptide showing b and y ions.

FIG. 11 depicts MASCOT results of ESI-MS/MS sequencing and database search identifying high molecular weight proteins found in embryo media.

### DETAILED DESCRIPTION OF THE INVENTION

For the purposes of promoting an understanding of the principles of the invention, reference will now be made to preferred embodiments and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

### I. Introduction

The invention provides a novel and non-invasive procedure to detect biomarkers in the pre-implantation embryo culture medium used to sustain individual IVF-derived human embryos before being transferred to the uterus. The method further includes correlating the detection and measurement of these biomarkers with a prognosis of developmental potential, which can include any or all of the successive stages of development beginning with embryo survival, implantation after uterine transfer and full term pregnancy. An IVF-derived human embryo diagnosed with favorable developmental potential has a high likelihood of surviving, successfully implanting after uterine transfer and/or leading to a full term pregnancy. An IVF-derived human embryo diagnosed with unfavorable developmental potential has a low likelihood of surviving, successfully implanting after uterine transfer or leading to a full term pregnancy. In another form of the invention, the method may facilitate making a prognosis or prediction of which IVF-derived embryos will have the highest likelihood of producing a successful implantation. In another form of the invention, the method may facilitate making a prognosis or prediction of which IVF-derived embryos will have the highest likelihood of resulting in a subsequent full term pregnancy after successful implantation.

The individual IVF-derived human embryos may be fresh embryos that are selected immediately following identification of pronuclear formation, or selected from any day of pre-implantation in vitro culture. The individual IVF-derived human embryos may also be cryopreserved embryos that were previously frozen (at any period after pronuclear formation, for example, during the cleavage and/or blastocyst stage) and are thawed and transferred. In another form of the invention, the method may facilitate making a prognosis or prediction of which IVF-derived cryopreserved embryos will have the highest likelihood of survival post-thaw. In another form of the invention, the method may facilitate making a prognosis or prediction of which IVF-derived cryopreserved embryos will have the highest likelihood of producing a successful implantation. In another form of the invention, the method may facilitate making a prognosis or prediction of which IVF-derived cryopreserved embryos will have the highest likelihood of producing a full term pregnancy.

The technique employs molecular profiling approaches that have extremely high sensitivity and specificity to detect and identify low concentrations of differentially expressed biomarkers in a biofluid or test sample, i.e., the human embryo culture medium. All the biomarkers are characterized by molecular weight. Biomarkers may be resolved from other proteins in a sample by chromatographic separation coupled with mass spectrometry. In preferred embodiments, the method of resolution involves MALDI-TOF-TOF and/or Surface-Enhanced Laser Desorption/Ionization (SELDI) mass spectrometry, in which the surface of the mass spectrometry probe comprises absorbents that bind to the biomarker. The invention employs the examination of defined secretory products (proteins or peptides) that characterize the individual embryo's viability, ability to implant following transfer to the uterus and ability to produce a full term pregnancy. These predictive proteins can be identified by methods such as SELDI, MALDI, ELISA, antibody capture, protein chips or diagnostic kits.

A biomarker is an organic biomolecule, the presence of which in a sample is used to determine the phenotypic status of the subject (e.g., a viable IVF-derived embryo with high developmental potential vs. an IVF-derived embryo with low developmental potential). In a preferred embodiment, the biomarker is differentially present in a sample taken from the culture medium supporting a single developing embryo of one phenotypic status (e.g., viable and implantation competent) as compared with another phenotypic status (e.g., not as robust and/or implantation incompetent). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. A single biomarker, or a combination of particular biomarkers, provides measures of relative risk or probability that a subject belongs to one phenotypic status or another. Therefore, they are useful as markers for disease (diagnostics), therapeutic effectiveness of a drug (theranostics), drug toxicity and in this present invention, identifying the quality and pregnancy potential of IVF-derived human embryos.

### II. Biomarkers Predictive of Embryo Developmental Potential

A. Biomarkers

This invention provides biomarkers that may be used to distinguish embryos with high developmental potential from embryos with less favorable developmental potential.

The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomarkers and not process limitations in the manner in which the biomarkers are discriminated.

The biomarkers were discovered and characterized using MALDI technology and/or SELDI technology. Negative control culture media (alone) samples were collected in addition to in vitro culture media samples from individual IVF-derived human embryo subjects that were chosen for uterine transfer (or selected for cryopreservation and subsequent uterine transfer) that led to a positive pregnancy, embryo subjects that were transferred and did not result in a positive implantation and embryo subjects that were not chosen for uterine transfer (or cryopreservation). The samples were fractionated by ion exchange chromatography, preferably by copper-coated immobilized metal affinity (IMAC-Cu) chromatography or WCX magnetic bead fractionation. Fractionated samples were applied to MALDI biochips and spectra of polypeptides in the samples were generated by time-of-flight mass spectrometry on a mass spectrometer. The resulting spectra obtained were analyzed by appropriate commercially available software. The mass spectra for each group were subjected to scatter plot analysis. A Mann-Whitney test analysis was employed to compare implantation competent embryos and implantation incompetent embryo groups for each protein cluster in the scatter plot, and proteins were selected that differed significantly (p<0.0001) between the two groups.

The biomarkers thus discovered are presented in Tables 1 and 2. The “ProteinChip assay” column of Table 2 refers to the type of biochip to which the biomarker binds and the wash conditions, as per the Example.

The biomarkers of this invention are characterized by their mass-to-charge ratio as determined by mass spectrometry. The mass-to-charge ratio of each biomarker is provided in Table 1 after the “M.” Thus, for example, M2454.00 has a measured mass-to-charge ratio of 2454.00. The mass-to-charge ratios are determined from mass spectra generated on any appropriate commercially available mass spectrometer. Preferably, the instrument will have a mass accuracy of about +/−0.3 percent. Additionally, the instrument will preferably have a mass resolution of about 400 to 1000 m/dm, where m is mass and dm is the mass spectral peak width at 0.5 peak height. The mass-to-charge ratio of the biomarkers was determined using appropriate commercially available software. Preferably, the software will assign a mass-to-charge ratio to a biomarker by clustering the mass-to-charge ratios of the same peaks from all the spectra analyzed, as determined by the mass spectrometer, taking the maximum and minimum mass-to-charge-ratio in the cluster, and dividing by two. Accordingly, the masses provided will reflect these specifications.

TABLE 1

| Marker No. | Mass (Da) |
|---|---|
| 1 | M2454.00 |
| 2 | M2648.00 |
| 3 | M2665.00 |
| 4 | M2684.00 |
| 5 | M2778.00 |
| 6 | M4093.00 |
| 7 | M37820.00 |
| 8 | M45873.00 |
| 9 | M282390.00 |
| 10 | M435170.00 |

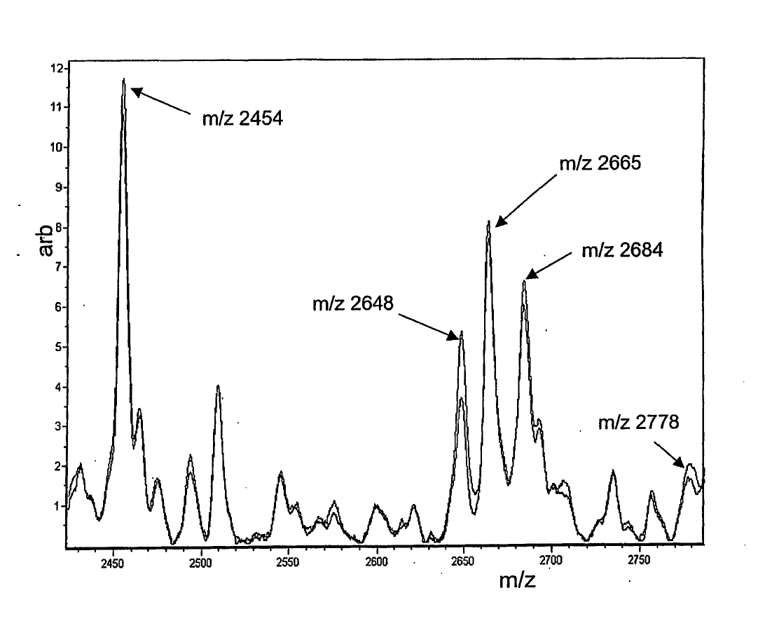

The biomarkers of this invention are further characterized by the shape of their spectral peak in time-of-flight mass spectrometry. Mass spectra showing peaks representing biomarkers listed in Table 1 are presented in FIG. 1.

TABLE 2

| | | | Up or down regulated | |
|---|---|---|---|---|
| Marker | | | in successful | |
| No. | Mass (Da) | P-Value | implantation | ProteinChip ® assay |
| 1 | M2454.00 | 0.000568 | down | WCX magnetic beads |
| 2 | M2648.00 | 0.000677 | down | WCX magnetic beads |
| 3 | M2665.00 | n.d | down | WCX magnetic beads |
| 4 | M2684.00 | 0.024 | down | WCX magnetic beads |
| 5 | M2778.00 | 0.000239 | down | WCX magnetic beads |
| 6 | M4093.00 | n.d. | n.d. | WCX magnetic beads |
| 7 | M37820.00 | n.d. | up | Isolated from embryo conditioned media |
| 8 | M45873.00 | n.d. | up | Isolated from embryo conditioned media |
| 9 | M282390.00 | n.d. | up | Isolated from embryo conditioned media |
| 10 | M435170.00 | n.d. | up | Isolated from embryo conditioned media |

The biomarkers of this invention may be further characterized by their binding properties on chromatographic surfaces. Most of the biomarkers bind to weak cation exchange magnetic beads after washing with 0.1% acetic acid.

Because the biomarkers of this invention are characterized by mass-to-charge ratio, binding properties and spectral shape, they may be detected by mass spectrometry without knowing their specific identity. However, if desired, biomarkers whose identity is not determined may be identified by, for example, determining the amino acid sequence of the polypeptides using MALDI-TOF-TOF. For example, a biomarker may be peptide-mapped with a number of enzymes, such as trypsin or V8 protease, and the molecular weights of the digestion fragments may be used to search databases for sequences that match the molecular weights of the digestion fragments generated by the various enzymes. Alternatively, protein biomarkers may be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein may be sequenced this way, or a sequence fragment may be subjected to database mining to find identity candidates.

“Biomarker” as used herein is defined as any molecule useful in differentiating IVF-derived embryos with developmental potential from those IVF-derived embryos with little or no developmental potential according to the invention. Presently preferred biomarkers according to the invention include proteins, protein fragments and peptides. The biomarkers may be isolated from a test sample, such as a sample of the in vitro embryo culture medium, used to support the ex vivo growth of an individual IVF-derived human embryo. In vitro embryo culture media, available from commercial sources, may be employed, and include P-1® MEDIUM (Pre-implantation Stage One) supplemented with 10% Serum Substitute Supplement (SSS™) from Irvine Scientific, Santa Ana, Calif. In another embodiment, the in vitro embryo culture media may be any media known in the art (Gardner, D. K., Lane M. and Schoolcraft W. B. Physiology and culture of the human blastocyst. Journal of Reproductive Immunology. 55(1):85-100 (2002); Gardner, D. K. and Schoolcraft, W. B. Culture and transfer of human blastocysts. Current Opinion in Obstetrics & Gynecology. 11 (3):307-311(1999)). It is standard practice to change the embryo culture media daily and it is therefore, otherwise normally discarded. The preferred source for detection of the biomarkers is the embryo culture medium. However, in another embodiment, the biomarkers may be detected in uterine secretions obtained by simple transcervical pipetting (or catheterization) in a mock cycle or a transfer cycle. In another embodiment, a protein or group of proteins predictive of implantation may be identified in patients' blood or serum at the time of, or after, the window of implantation. The biomarkers may be isolated by any method known in the art, based on both their mass and their binding characteristics. For example, a test sample comprising the biomarkers may be subject to chromatographic fractionation, as described herein, and subject to further separation by, e.g., acrylamide gel electrophoresis. Knowledge of the identity of the biomarker also allows their isolation by immunoaffinity chromatography. As used herein, the term “detecting” includes determining the presence, the absence, the quantity, or a combination thereof, of the biomarkers. The quantity of the biomarkers may be represented by the peak intensity as identified by mass spectrometry, for example, or concentration of the biomarkers.

The biomarker is typically differentially present in test samples from IVF-derived embryos with favorable developmental potential relative to those with little or no developmental potential. However, some biomarkers, while not being differentially expressed between two classes may, nevertheless, be classified as a biomarker according to the present invention to the extent that they are significant in delineating subsets of groups in a classification tree.

The differential expression, such as the over- or under-expression, of selected biomarkers relative to IVF-derived negative control embryos with little or no developmental potential may be correlated to developmental potential. By differentially expressed, it is meant herein that the biomarker(s) may be found at a greater or lesser quantity in the test sample compared to a negative control, or that it may be found at a higher frequency (e.g. intensity) in one or more test samples. For example, the underexpression of the about 2454±5, 2648±5.3, 2665±5.3, 2684±5.4, 2778±5.6 and 4093±8.2 Dalton biomarkers, or the underexpression of the about 2454±5, 2648±5.3, 2665±5.3, 2684±5.4 and 2778±5.6 Dalton biomarkers, relative to control embryos with little or no developmental potential, may be correlated with the diagnosis of favorable developmental potential. Furthermore, the overexpression of the about 37820±76, 45873±92, 282390±565 and 435170±870 Dalton biomarkers, relative to embryos with little or no developmental potential, may be correlated with a diagnosis of favorable developmental potential. By control, it is meant herein that a control test sample or control test samples are those characterized test samples which correlate to known developmental potential outcomes; those with favorable developmental potential (or positive controls) and those with little or no developmental potential at any stage (negative controls), i.e., implantation failure or full term pregnancy failure. By blank control, it is meant herein that a blank control test sample is a test sample or test samples which have not come into contact with an IVF-derived human embryo.

B. Modified Forms of Proteins as Biomarkers

It has been found that proteins frequently exist in a sample in a plurality of different forms characterized by detectably different masses. These forms may result from pre-translational modifications, post-translational modifications or both. Pre-translational modified forms include allelic variants, splice variants and RNA editing forms. Post-translationally modified forms include forms resulting from, among other things, proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of any biomarker of this invention also may be used, themselves, as biomarkers. In certain cases the modified forms may exhibit better discriminatory power in diagnosis than the specific forms set forth herein.

Modified forms of a biomarker may be initially detected by any methodology that can detect and distinguish the modified form of the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, interacting fission proteins, or aptamers that recognize the biomarker and modified forms of it. This method may also result in the capture of protein interactors that are bound to the proteins or that are otherwise recognized by antibodies and that, themselves, can be biomarkers. Preferably, the biospecific capture reagents are bound to a solid phase. Then, the captured proteins may be detected by MALDI or SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They may employ, for example, bifunctional linking agents, or the solid phase can be derivatized with a reactive group, such as an epoxide or an imidizole, that will bind the molecule on contact. Biospecific capture reagents against different target proteins may be mixed in the same place, or they may be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies may be used to detect the protein clusters. However, the biospecific capture reagents must be specifically directed toward the members of a cluster in order to differentiate them.

In yet another embodiment, the surfaces of biochips may be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form may be used as a biomarker in any of the methods of this invention. At this point, detection of the modified form may be accomplished by any specific detection methodology including affinity capture followed by mass spectrometry, or traditional immunoassay directed specifically to the modified form. Immunoassay requires biospecific capture reagents, such as antibodies, to capture the analytes. Furthermore, the assay must be designed to specifically distinguish a protein and modified forms of the protein. This may be done, for example, by employing a sandwich assay in which one antibody captures more than one form and second, distinctly labeled antibodies, specifically bind the various forms, thereby providing distinct detection of them. Antibodies may be produced by immunizing animals with the biomolecules. This invention contemplates traditional immunoassays including, for example, sandwich immunoassays including ELISA or fluorescence-based immunoassays, as well as other enzyme immunoassays, biochips of selected proteins such as a microarray and diagnostic kits.

### III. Detection of Embryo Developmental Potential Biomarkers

The biomarkers of this invention may be detected by any suitable method. Detection paradigms that may be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. Suitable biochips include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications; U.S. Pat. No. 6,225,047, PCT International Publication No. WO 99/51773, U.S. Pat. No. 6,329,209, PCT International Publication No. WO 00/56934 and U.S. Pat. No. 5,242,828.

A. Detection by Mass Spectrometry

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

1. SELDI

A preferred mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent may be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA-protein conjugate). In certain instances, the biospecific adsorbent may be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with carboimidizole, expoxide) and PG-20 (protein G coupled through carboimidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitriloacetic acid functionalities that adsorb transition metal ions, such as copper, nickel, zinc, and gallium, by chelation. Preactivated ProteinChip arrays have carboimidizole or epoxide functional groups that can react with groups on proteins for covalent binding.

Such biochips are further described in; U.S. Pat. No.6,579,719 (Hutchens and Yip, “Retentate Chromatography,” Jun. 17, 2003), PCT International Publication No. WO 00/66265 (Rich et al., “Probes for a Gas Phase Ion Spectrometer,” Nov. 9, 2000); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application No. US 2003/0218130 A1 (Boschetti et al., “Biochips With Surfaces Coated With Polysaccharide-Based Hydrogels,” Apr. 14, 2003) and U.S. Patent Application No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow biomarker or biomarkers that may be present in the sample to bind to the adsorbent. After an incubation period, the substrate is washed to remove unbound material. Any suitable washing solutions may be used; preferably, aqueous solutions are employed. The extent to which molecules remain bound may be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition may be a co-polymer of a-cyano-4-methacryloyloxycinnamic acid and acrylate. In another embodiment, the composition is a co-polymer of a-cyano-4-methacryloyloxycinnamic acid, acrylate and 3-(tri-ethoxy)silyl propyl methacrylate. In another embodiment, the composition is a co-polymer of a-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety, which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

2. Other Mass Spectrometry Methods

In another mass spectrometry method, the biomarkers may be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

3. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. These time-of-flight data are then subject to data processing. Data processing typically includes TOF-to-M/Z transformation to generate a mass spectrum, baseline subtraction to eliminate instrument offsets and high frequency noise filtering to reduce high frequency noise.

Data generated by desorption and detection of biomarkers may be analyzed with the use of a programmable digital computer. The computer program analyzes the data to indicate the number of biomarkers detected, and optionally the strength of the signal and the determined molecular mass for each biomarker detected. Data analysis may include steps of determining signal strength of a biomarker and removing data deviating from a predetermined statistical distribution. For example, the observed peaks may be normalized, by calculating the height of each peak relative to some reference. The reference may be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection may be done visually, but software is commercially available that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of commercially available software, Ciphergen's ProteinChip®, clusters all peaks appearing in the various spectra within a defined mass range, and assigns a mass (M/Z) to all the peaks that are near the mid-point of the mass (M/Z) cluster.

Software used to analyze the data may include code that applies an algorithm to the analysis of the signal to determine whether the signal represents a peak in a signal that corresponds to a biomarker according to the present invention. The software also may subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

4. General Protocol for SELDI Detection of Biomarkers for Embryo Developmental Potential

A preferred protocol for the detection of the biomarkers of this invention is as follows. For SELDI the biological sample to be tested, e.g., embryo culture medium, the medium is placed on the chip and non-specific peptides-proteins are washed away so that the profile obtained represents a fraction of the proteins present in the sample that specifically bind to the chip. There is neither previous prefractionation nor centrifugation of the sample.

Another preferred protocol for the detection of the biomarkers of this invention is as follows: For MALDI: Prefractionation of the sample is performed on magnetic beads before the MALDI and then the peptides and proteins are eluted from the beads and the material is put on the target MALDI plate. For both methods, fluids are initially stored at minus 80° C. and then used for MALDI or SELDI without centrifugation or other interventions.

The sample to be tested (preferably pre-fractionated) is then contacted with an affinity capture chip comprising a cation exchange adsorbent (preferably a WCX ProteinChip array) or an IMAC adsorbent (preferably an IMAC3 ProteinChip array). The chip is washed with a buffer that will retain the biomarker while washing away unbound molecules. The biomarkers are detected by laser desorption/ionization mass spectrometry.

Alternatively, if antibodies that recognize the biomarker are available, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

B. Detection by Immunoassay

In another embodiment, the biomarkers of this invention may be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### IV. Determination of Embryo Developmental Potential

A. Single Markers

The biomarkers of the invention may be used in diagnostic tests to assess developmental potential in an individual human embryo, e.g., to determine the probability of a successful implantation and subsequent live birth following uterine transfer. Based on this status, further procedures may be indicated, including additional diagnostic tests or therapeutic procedures or regimens.

The power of a diagnostic test to correctly predict developmental potential is commonly measured as the sensitivity of the assay, the specificity of the assay or the area under a receiver perated characteristic (“ROC”) curve. Sensitivity is the percentage of true positives (i.e.; successful implantation) that are predicted by a test to be positive, while specificity is the percentage of true negatives (i.e.; failed implantation) that are predicted by a test to be negative. A ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive and represents the capacity of the test (single biomarker, a combination of biomarkers or a protein profile) to accurately determine successful implantation of a given embryo or group of embryos (fresh or frozen thawed). Negative predictive value is the percentage of actual negatives that test as negative and represent the capacity of the test to determine failed implantation of a given embryo or group of embryos.

Each biomarker listed in Table 1 is individually useful in aiding in a diagnosis of developmental potential. The method involves, first, detecting the selected biomarker in a subject sample using the methods described herein, e.g., capture on a SELDI biochip followed by detection by mass spectrometry and, second, comparing the measurement with a diagnostic amount or cut-off that distinguishes a favorable developmental potential diagnosis from a less favorable developmental potential diagnosis. The diagnostic amount represents a measured amount of a biomarker above which or below which a subject is classified as having a particular developmental potential. For example, if the biomarker is up-regulated or overexpressed for embryos that produced a successful implantation compared to embryos that did not produce a successful implantation, then a measured amount above the diagnostic cutoff provides a diagnosis of favorable implantation competence or potential. Alternatively, if the biomarker is down-regulated or underexpressed for embryos that produced a successful implantation compared to embryos that did not produce a successful implantation, then a measured amount below the diagnostic cutoff provides a diagnosis of favorable implantation competence or potential. As is well understood in the art, by adjusting the particular diagnostic cut-off used in an assay, one may increase sensitivity or specificity of the diagnostic assay depending on the preference of the diagnostician. The particular diagnostic cut-off can be determined, for example, by measuring the amount of the biomarker in a statistically significant number of test samples from subjects with the different developmental potential outcomes, and drawing the cut-off to suit the diagnostician's desired levels of specificity and sensitivity.

B. Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test.

The protocols described in the Example below were used to generate 1404 mass spectra from 702 media samples, 157 of which were from IVF-derived embryos that were transferred and produced a positive pregnancy. The peak masses and heights were abstracted into a discovery data set. This data set was used to construct genetic algorithms. Genetic algorithms can be generated by employing classification and regression tree analysis (CART). For each subset, CART will generate a best or near best decision tree to classify a sample as implantation competent or implantation incompetent. Among the many decision trees generated by CART, the preferred tree will have excellent sensitivity and specificity in distinguishing the most viable embryos with the highest pregnancy potential vs those that failed to implant following uterine transfer.

1. Decision Tree

In one embodiment, particular biomarkers may be useful in combination to classify favorable developmental potential vs. embryos with unfavorable developmental potential. Preferably, the combination of these groupings makes up a single classification tree for a diagnosis of favorable developmental potential or unfavorable developmental potential. However, the present invention contemplates the use of these individual groupings alone or in combination with other groupings to aid in the diagnosis of developmental potential. Thus, one or more of such groupings, preferably two or more, or more preferably, all of these groupings aid in the diagnosis.

2. SDS Algorithm

The same data set that may be employed in the previously described CART analysis may be used with the multi-stage Statistical Classification Strategy (SCS) (Institute for Biodiagnostics, National Research Council Canada, Winnipeg, MB, Canada). SCS involves feature (mass peak) selection with a two-stage exhaustive search, using a wrapper approach. The classifier used in the wrapper may be the simple linear discriminant with leave-one-out (LOO) cross validation. Once the optimally discriminatory peaks are found, the final classifier may be obtained with a bootstrap-inspired approach.

C. Subject Management

In certain embodiments of the methods of qualifying developmental potential status, the methods further comprise managing subject treatment based on the status. Such management includes the actions of the physician or clinician subsequent to determining developmental potential status. For example, if a physician makes a diagnosis of unfavorable developmental potential, then a certain regime of treatment might follow for future egg harvest including use of a different ovarian stimulation protocol (type of medications, dosages, duration of treatment), use of different culture conditions (culture media or media supplementation); and if the problem persists after modifications then it could be recommended to consider use of oocyte or sperm donation or other alternative approaches.

Additional embodiments of the invention relate to the communication of assay results or diagnoses or both to technicians, physicians or patients, for example. In certain embodiments, computers may be used to communicate assay results or diagnoses or both to interested parties, e.g., physicians and their patients. In some embodiments, the assays may be performed or the assay results analyzed in a country or jurisdiction that differs from the country or jurisdiction to which the results or diagnoses are communicated.

In a preferred embodiment of the invention, a diagnosis based on the presence or absence in a test subject of any the biomarkers is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software, which is familiar to artisans skilled in telecommunications. One example of a healthcare-oriented communications system is described in U.S. Pat. No. 6,283,761; however, the present invention is not limited to methods that utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### V. Generation of Classification Algorithms for Qualifying Embryo Developmental Potential

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., implantation competent versus implantation incompetent).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention may be embodied by computer code that is executed or used by a digital computer. The computer code may be stored on any suitable computer readable media including optical or magnetic disks, sticks, tapes, etc., and may be written in any suitable computer programming language including C, C++, visual basic, etc.

The learning algorithms described above are useful both for developing classification algorithms for the biomarkers already discovered, and for finding new biomarkers for implantation competence. The classification algorithms, in turn, form the base for diagnostic tests by providing diagnostic values (e.g., cut-off points) for biomarkers used singly or in combination.

### VI. Kits for Detection of Biomarkers for Embryo Developmental Potential

In another aspect, the present invention provides kits for qualifying embryo implantation status, in which kits are used to detect biomarkers according to the invention. In one embodiment, the kit comprises one or more container means, preferably comprising a solid support, such as a chip, a microtiter plate or a bead or resin having a capture reagent attached thereon, wherein the capture reagent binds a biomarker of the invention. Thus, for example, the kits of the present invention can comprise MALDI or mass spectrometry probes for SELDI such as ProteinChip® arrays. In the case of biospecific capture reagents; the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

In yet another embodiment, the kit can comprise components for establishing one or more control population values or ranges.

### VII. Use of Biomarkers for Embryo Developmental Potential in Screening Assays

The methods of the present invention have other applications as well. For example, the biomarkers may be used to screen for compounds that modulate the expression of the biomarkers in vitro or in vivo, which compounds in turn may be useful in treating or preventing implantation incompetence in embryos. In another example, the biomarkers may be used to monitor the response to treatments for implantation competence. Information obtained from the predictive biomarkers identified in this application could potentially be used to develop novel contraceptive strategies. For example, protein markers found in the embryo culture media, patient's blood or endometrial fluids could be antagonized or eliminated systemically or locally

Thus, for example, the kits of this invention could include a solid substrate having a hydrophobic function, such as a protein biochip (e.g., a Ciphergen H50 ProteinChip array, e.g., ProteinChip array) and a sodium acetate buffer for washing the substrate, as well as instructions providing a protocol to measure the biomarkers of this invention on the chip and to use these measurements to diagnose implantation incompetence.

Compounds suitable for therapeutic testing may be screened initially by identifying compounds that interact with one or more biomarkers listed in Table 1. By way of example, screening might include recombinantly expressing a particular biomarker listed in Table 1, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers listed in Table 1, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table 1 may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers in Table 1 may also be measured. For example, the self-assembly of a multi-protein complex which includes one of the biomarkers of Table 1 may be monitored by spectroscopy in the presence or absence of a test compound. Alternatively, if the biomarker is a non-enzymatic enhancer of transcription, test compounds that interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table 1 may be administered to patients who may develop implantation incompetent eggs and/or sperm; i.e., those eggs and/or sperm that result in implantation incompetent embryos. In addition, as the embryonic genome is activated at the 4-8 cell stage in the human, proteins expressed at this time may be related to eggs or sperm or may be respective of the resulting embryo. In the latter case, test compounds capable of modulating the activity of any of the biomarkers may be administered to the embryo. For example, the administration of a test compound which, increases the activity of a particular biomarker, may decrease the risk of the development of implantation incompetent eggs and/or sperm in a patient if the activity of the particular biomarker in vivo prevents the accumulation of proteins for implantation incompetence. Conversely, the administration of a test compound which decreases the activity of a particular biomarker may decrease the risk of developing implantation incompetent eggs and/or sperm in a patient if the increased activity of the biomarker is responsible, at least in part, for the onset of implantation incompetent eggs and/or sperm.

In an additional aspect, the invention may provide a method for identifying compounds useful for the treatment of implantation incompetent eggs and/or sperm, which are associated with specific levels of modified forms of any of the biomarkers of Table 1. For example, in one embodiment, cell extracts or expression libraries may be screened for compounds that catalyze the cleavage of a full-length biomarker to form a truncated form of the biomarker. In one embodiment of such a screening assay, cleavage of a biomarker may be detected by attaching a fluorophore to the biomarker, which remains quenched when the biomarker is uncleaved but which fluoresces when the protein is cleaved. Alternatively, a version of full-length biomarker modified so as to render the amide bond between amino acids x and y uncleavable may be used to selectively bind or “trap” the cellular protease which cleaves a full-length biomarker at that site in vivo. Methods for screening and identifying proteases and their targets are well documented in the scientific literature, e.g., in Lopez-Ottin et al. (Nature Reviews, 3:509-519 (2002)).

In yet another embodiment, the invention provides a method for treating or reducing the incidence or likelihood of producing developmental incompetent eggs and/or sperm, which is associated with the increased or decreased levels of any of the biomarkers of Table 1. For example, after one or more proteins have been identified which inhibit or activate a biomarker, combinatorial libraries may be screened for compounds that inhibit or activate the identified proteins. Methods of screening chemical libraries for such compounds are well known in art. See, e.g., Lopez-Otin et al. (2002). Alternatively, inhibitory compounds may be intelligently designed based on the structure of any of the biomarkers of Table 1.

Compounds which impart a truncated biomarker with the functionality of a full-length biomarker are likely to be useful in treating conditions, such as production of implantation incompetent eggs and/or sperm, which are associated with the truncated form of the biomarker. Therefore, in a further embodiment, the invention may provide methods for identifying compounds that increase the affinity of a truncated form of any of the biomarkers of Table 1 for its target proteases. For example, compounds may be screened for their ability to impart a truncated biomarker with the protease inhibitory activity of the full-length biomarker. Test compounds capable of modulating the inhibitory activity of a biomarker or the activity of molecules that interact with a biomarker may then be tested in vivo for their ability to slow or stop the progression of the production of implantation incompetent eggs and/or sperm in a subject.

At the clinical level, screening a test compound includes obtaining samples from test subjects before and after the subjects have been exposed to a test compound. The levels in the samples of one or more of the biomarkers listed in Table 1 may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table 1 may be measured directly by Western blot using radio- or fluorescently-labeled antibodies that specifically bind to the biomarkers. Alternatively, changes in the levels of MRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells, which express, or are capable of expressing, one or more of the biomarkers of Table 1, may be contacted with test compounds. Subjects who have been treated with test compounds may be routinely examined for any physiological effects that may result from the treatment. In particular, the test compounds may be evaluated for their ability to decrease failed pregnancy likelihood in a subject. Alternatively, if the test compounds are administered to subjects who have previously been diagnosed with the production of implantation incompetent eggs and/or sperm, test compounds may be screened for their ability to decrease or stop further failed pregnancy occurrences.

The invention is described in greater detail by way of specific examples. The examples are offered for illustrative purposes, and are not intended to limit the invention in any manner. Those of skill in the art will readily recognize a variety of non-critical parameters that can be changed or modified to yield essentially the same results.

### EXAMPLES

### Example 1

### Discovery of Biomarkers for Qualifying Embryo Developmental Potential

### Embryo Media Samples

IVF-derived human embryo subjects were generated from patients seen at the Jones Institute for Reproductive Medicine in Norfolk, Va. In all cases, patient consent was obtained according to the regulations for human subject protection of each institution. The embryo media samples (n=702) were obtained from in vitro embryo culture medium, which is used to sustain individual IVF-derived human embryos. Following identification of pronuclear formation (standard care of IVF), IVF-derived embryos were cultured individually in a micro droplet using oil. The media sample was taken on either day 2 or day 3 of the in vitro culture period, prior to uterine transfer of the embryo or cryopreservation of the embryo for potential future uterine transfer. Embryo media samples (n=356) were obtained from day 2 embryos including those chosen for uterine transfer (n=250) or those that were not selected for transfer (n=106). Embryo media samples (n=346) were obtained from day 3 embryos including those chosen for uterine transfer (n=243) or those that were not selected for transfer (n=103). Samples of the same embryo culture medium alone, which were never in contact with an IVF-derived human embryo, were included as negative controls. The embryo media samples were aliquoted and frozen at −80° C. until thawed specifically for MALDI analysis.

### MALDI Processing of Embryo Media Samples

10 μl of each embryo culture media sample was run via WCX magnetic bead fractionation. The range of proteins analyzed was 500 to 9000 Da. To identify profiles predictive of successful implantation, fresh embryo transfers (vs. cryopreserved embryos) were analyzed first. The resulting captured peptides and proteins were run on the Bruker Daltonics Ultraflex TOF mass spectrometer. Each sample was spotted in duplicate on the MALDI target. The spectra were analyzed to evaluate protein peaks differentially expressed between positive control embryos that led to a successful implantation, and those negative controls that did not, from day 2 and day 3 of in vitro embryo growth. Genetic algorithms constructed to classify those embryos that produced a successful pregnancy were constructed for day 2 (Table 3) and day 3 media samples (Table 4). The classification algorithm of Table 3 is based on 11 peaks; recognition capability: pregnant: 69.29%, not pregnant: 91.74%; cross validation: pregnant: 39.22%, not pregnant: 82.9%.

TABLE 3

| m/z | p-value |
|---|---|
| 2084.25 | 0.653 |
| 2110.71 | 0.000000018 |
| 2235.17 | 0.817 |
| 2360.27 | 0.029 |
| 2381.52 | 0.000057 |
| 2648.42 | 0.0000022 |
| 2756.66 | 0.089 |
| 2917.09 | 0.199 |
| 2940.00 | 0.056 |
| 3354.65 | 0.000022 |
| 4096.11 | 0.00000002 |

TABLE 4

| m/z | p-value |
|---|---|
| 2454.67* | 0.00059 |
| 2575.03 | 0.492 |
| 2648.20 | 0.00068 |
| 2684.72 | 0.024 |
| 2705.26 | 0.0026 |
| 2778.87 | 0.00024 |
| 2984.35 | 0.492 |
| 3286.91 | 0.479 |
| 3407.21 | 0.522 |
| 5757.97 | 0.877 |
| 9099.75 | 0.086 |

The classification algorithm of Table 4 is based on 11 peaks; recognition capability: pregnant: 88.5%, not pregnant: 100%; cross validation: pregnant: 40.6%, not pregnant: 72.1%.

Since the transfer of multiple embryos resulting in only one positive pregnancy may confound the data, a subset was identified in which an equal number of embryos transferred was equal to the number of implanted embryos or positive pregnancies. This subset of samples from day 2 (n=20) and day 3 (n=20) was tested as unknown samples with the best performing genetic algorithm developed from the entire sample set. A total of 23/40 (57.5%) of day 2 samples classified correctly as implanting successfully while 27/40 (67.5%) of day 3 samples classified correctly as implanting successfully. The classification rate for successfully implanted positive control embryos was improved to 67.5% for day 3 embryos and 57.5% for day 2 embryos.

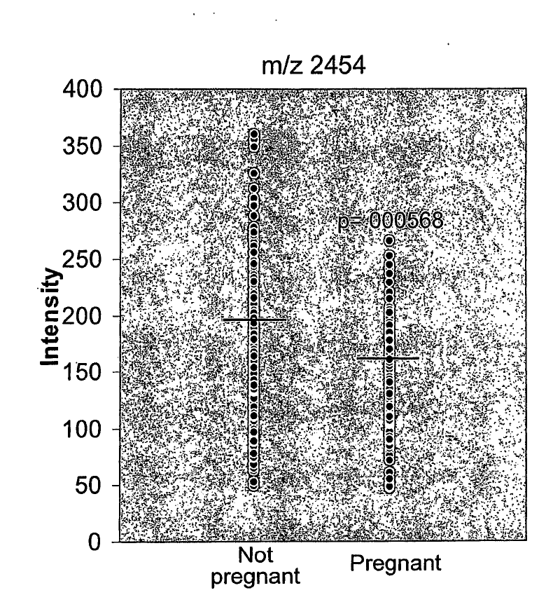

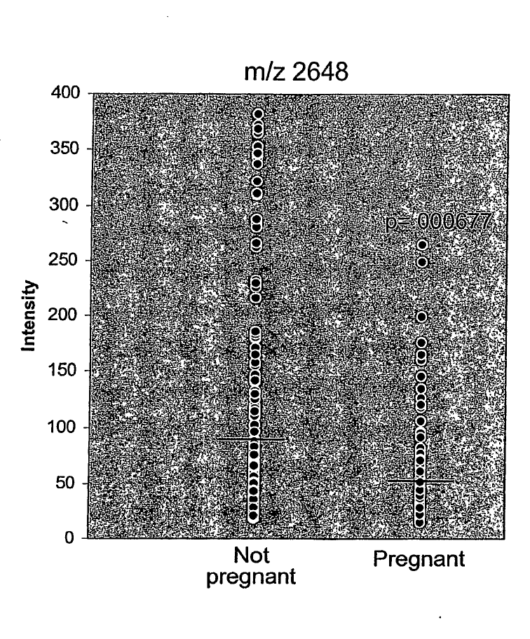

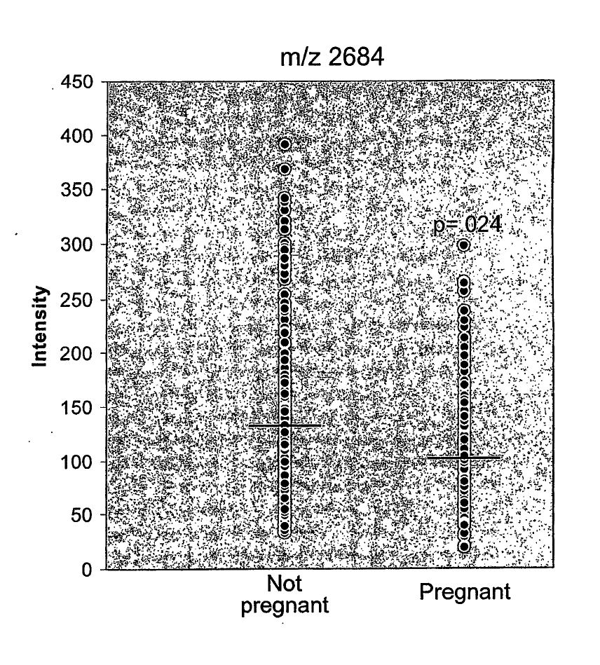

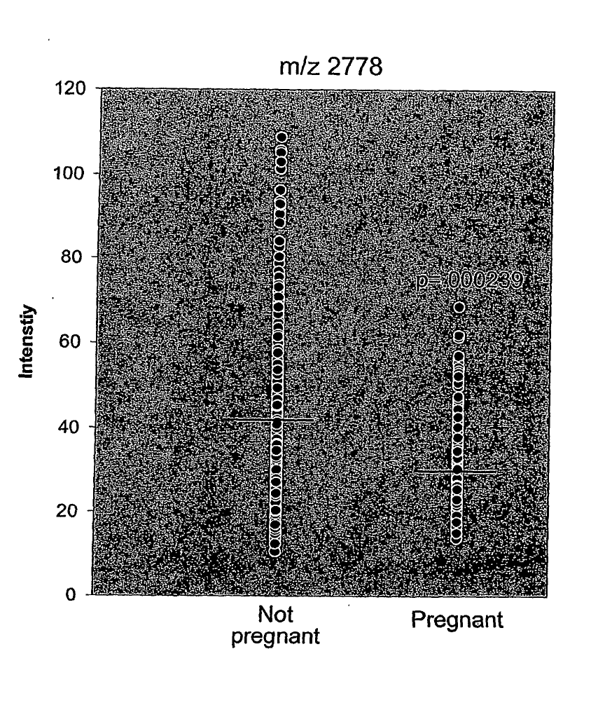

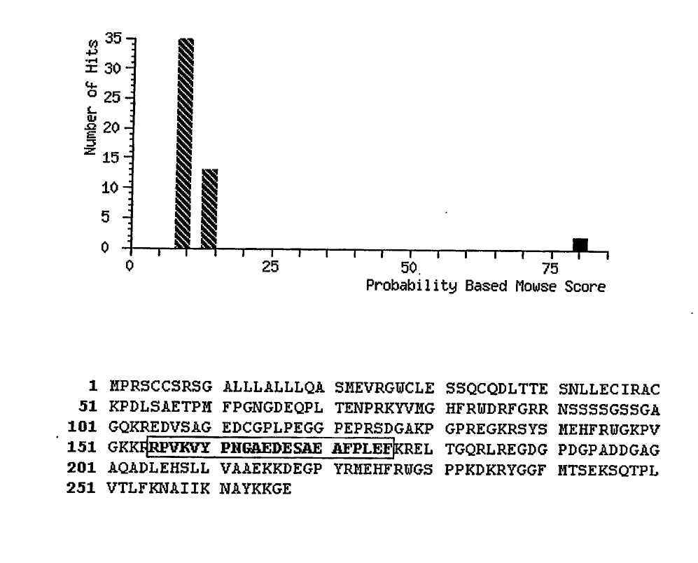

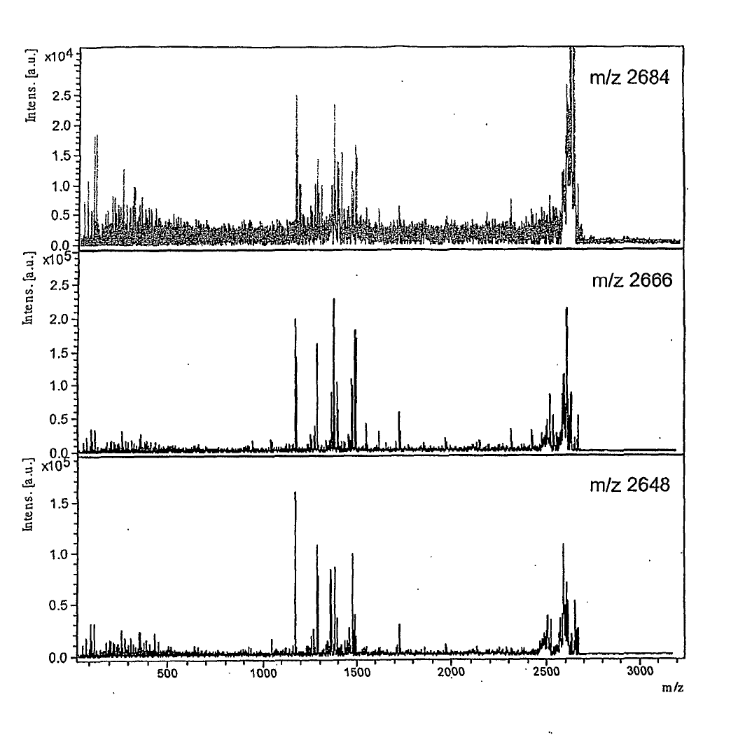

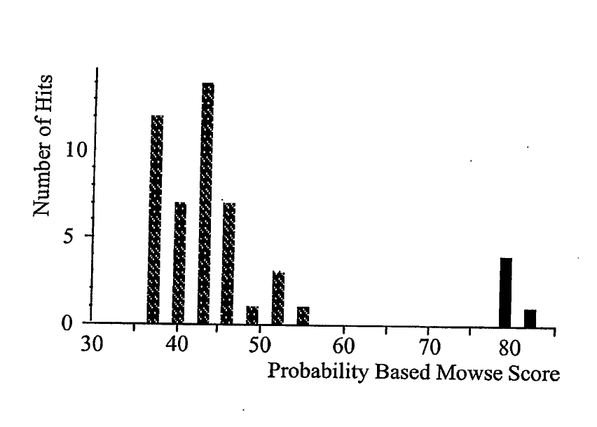

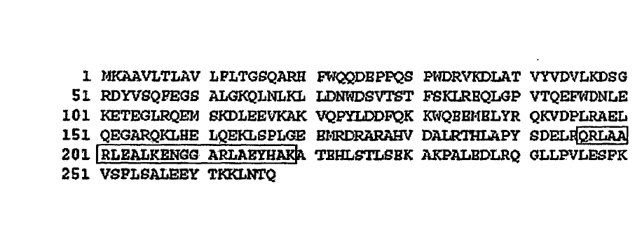

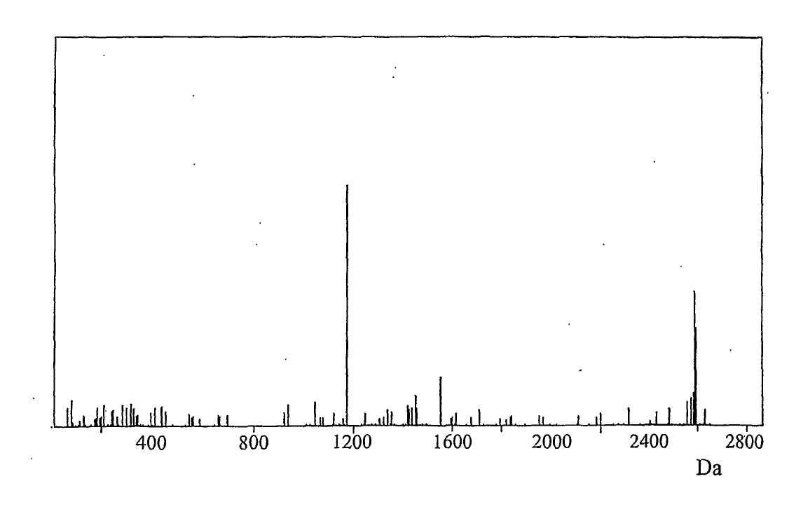

As shown in FIGS. 1-5, peaks at m/z 2454, 2648, 2665, 2684 and 2778 all show reduced expression in the embryo media samples from positive control embryos that produced a positive pregnancy. The embryos may preferentially take up these peptides or proteins from the growth media. The peak at m/z 2454 has been identified as potentially two proteins: Opiomelanocortin (FIG. 6) via direct post source fragmentation of LIFT-MS and Late cornified envelope protein from a pooled sample run on ESI-MS. As confirmed by LIFT sequencing, the peaks at 2648 and 2684 are the same protein as m/z 2665 with the loss or gain of one water molecule (18 Da), since the LIFT spectra are identical (FIG. 7). These peaks were identified as a fragment Apolipoprotein A1 (FIGS. 8-10 ). To date, the peak at 2778 has not been identified.

Further profiling experiments using embryo media from negative control deselected embryos (embryos not chosen for transfer or cryopreservation after microscopic examination by an embryologist) were performed to determine if the same peaks found to be discriminatory in the study using media samples from transferred embryos were also important in discriminating deselected embryos from transferred or cryogenically preserved embryos. As shown in Table 5, an algorithm was developed to classify deselected vs. transferred embryos. The classification algorithm of Table 5 is based on 7 peaks; cross validation: deselected: 20%, number of iterations: 10, overall; 57.4%, class 1: 80.06%, class 2: 34.74%. Two of these peaks (m/z 2646 and m/z 2662) were statistically significant in the previously developed algorithms from the initial profiling study that have now been identified as a fragment of Apolipoprotein A1.

TABLE 5

| m/z | p-value |
|---|---|
| 1640.97 | 0.00108 |
| 1658.15 | 0.0955 |
| 1847.8 | 0.826 |
| 2646.55 | 0.0169 |
| 2662.61 | 3.3 × 10⁻¹⁰ |
| 3252.05 | 4.37 × 10⁻⁹ |
| 3640.7 | 0.114 |

Another algorithm (Table 6) was developed which classifies negative control deselected vs. cryopreserved embryo media. Thus, the reduced expression of the common peaks identified as a fragment of Apolipoprotein A1 (2646+2 Da, 2662+3 Da, 2683+2 Da); between both profiling studies represent important markers for the classification of viable embryos that produce a pregnancy. The classification algorithm of Table 6 is based on 8 peaks; cross validation: deselected: 20%, number of iterations: 10, overall; 59.18%, class 1: 82.2%, class 2: 36.17%.

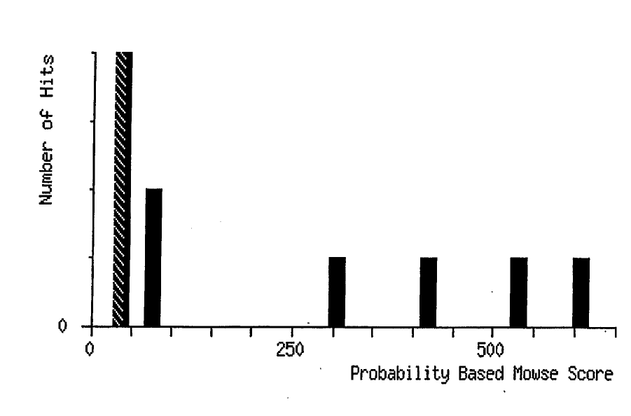

Additional studies for the discovery of high molecular weight proteins present in the embryos culture medium have yielded the identification of additional biomarkers shown in Table 1 as Markers 5-8; 37820±76, 45873±92, 282390±565 and 435170±870 Daltons. The MS/MS results are shown in FIG. 11. These proteins were present in embryo culture media samples, where the embryo was chosen for cryopreservation or uterine transfer, and were not present in the control (blank) embryo culture media alone and therefore may represent important proteins secreted by viable embryos.

TABLE 6

| m/z | p value |
|---|---|
| 1641.01 | 0.00177 |
| 1658.04 | 0.000168 |
| 2646.61 | 6.7 × 10⁻⁶ |
| 2662.56 | 2.5 × 10⁻¹⁴ |
| 2683.01 | 7.4 × 10⁻¹⁵ |
| 4093.26 | 0.000138 |
| 4498.17 | 2.5 × 10⁻¹² |
| 4825.99 | 5.7 × 10⁻⁵ |

The peak at m/z 37820 has been identified as MOS_HUMAN (Oocyte maturation gene): which is expressed specifically in testis during spermatogenesis. It is also expressed in the placenta. The MOS genes with the highest transforming activity efficiently induce maturation in oocytes and mimic Cytostatic factor (CSF) by causing mitotic cleavage arrest in embryos. The ability to induce oocyte maturation might be responsible to govern the growth and development of the human oocyte and pre-implantation embryo.

The peak at m/z 45873 has been identified as Zygote Arrest 1 gene (ZAR-1), which is an oocyte-specific maternal-effect marker gene. It plays a vital role in the oocyte to embryo transition. It is essential for the beginning of embryo development (discovered in mice). It is believed to be one of the key regulators of successful pre-implantation development in domestic animals and humans.

The peak at m/z 282390 has been identified as Hornerin, which is a novel member of the “fused gene” -type, cornified envelope precursor protein family. It is expressed specifically in embryonic skin. High levels of expression have been documented in adult forestomach while lower levels have been found in the tongue and oesophagus.

The peak at m/z 435170 has been identified as Filaggrin. Filaggrins are filament-associated proteins that bind to keratin fibers in epidermal cells.

While the invention has been illustrated and described in the figures and foregoing description, the same is to be considered as illustrative and not restrictive in character, it being understood that only the preferred embodiments have been shown and described and that all changes and modifications that come within the spirit of the invention are desired to be protected. In addition, all references and patents cited herein are indicative of the level of skill in the art and hereby incorporated by reference in their entirety.

What is claimed is:

1. A method for aiding in a diagnosis of developmental potential in an IVF-derived human embryo comprising:
a) culturing said IVF-derived human embryo in embryo culture media;
b) obtaining a test sample from said embryo culture media;
c) detecting the quantity of at least one biomarker in said test sample, said at least one biomarker selected from the group consisting of biomarkers having a molecular weight of about 2454±5, 2648±5.3, 2665±5.3, 2684±5.4, 2778±5.6, 4093±8.2, 37820±76, 45873±92, 282390±565 and 435170±870 Daltons;
d) comparing the quantity of said at least one biomarker in said test sample to the quantity of the same said at least one biomarker in a control sample known to lack favorable developmental potential;
e) wherein the differential expression of said at least one biomarker in said test sample relative to said control sample is correlated with a diagnosis of favorable developmental potential.

2. A method for aiding in a diagnosis of developmental potential in an IVF-derived human embryo comprising:
a) culturing said IVF-derived human embryo in embryo culture media;
b) obtaining a test sample from said embryo culture media;
c) detecting the quantity of at least one biomarker in said test sample, said at least one biomarker selected from the group consisting of biomarkers having a molecular weight of about 2454±5, 2648±5.3, 2665±5.3, 2684±5.4, 2778±5.6 and 4093±8.2 Daltons;
d) comparing the quantity of said at least one biomarker in said test sample to the quantity of the same said at least one biomarker in a control sample known to lack favorable developmental potential;
e) wherein the underexpression of said at least one biomarker in said test sample relative to said control sample is correlated with a diagnosis of favorable developmental potential.

3. A method for aiding in a diagnosis of developmental potential in an IVF-derived human embryo comprising:
a) culturing said IVF-derived human embryo in embryo culture media;
b) obtaining a test sample from said embryo culture media;
c) detecting the quantity of at least one biomarker in said test sample, said at least one biomarker selected from the group consisting of biomarkers having a molecular weight of about 2454±5, 2648±5.3, 2665±5.3, 2684±5.4 and 2778±5.6 Daltons;
d) comparing the quantity of said at least one biomarker in said test sample to the quantity of the same said at least one biomarker in a control sample known to lack favorable developmental potential;
e) wherein the underexpression of said at least one biomarker in said test sample relative to said control sample is correlated with a diagnosis of favorable developmental potential.

4. A method for aiding in a diagnosis of developmental potential in an IVF-derived human embryo comprising:
a) culturing said IVF-derived human embryo in embryo culture media;
b) obtaining a test sample from said embryo culture media;
c) detecting at least one biomarker in said test sample, said at least one biomarker selected from the group consisting of biomarkers having a molecular weight of about 37820±76, 45873±92, 282390±565 and 435170±870 Daltons;
d) wherein the detection of said at least one biomarker in said test sample is correlated with a diagnosis of favorable developmental potential.

5. The method of any of claims 1 to 4, wherein said developmental potential being diagnosed is implantation potential.

6. The method of any of claims 1 to 4, wherein said developmental potential being diagnosed is full term pregnancy potential.

7. The method of any of claims 1 to 4, wherein said IVF-derived human embryo is cryopreserved.

8. The method of any of claims 1 to 4, wherein said IVF-derived human embryo is not cryopreserved.

9. The method of any of claims 1 to 4, wherein said test sample is obtained at culture day 2.

10. The method of any of claims 1 to 4, wherein said test sample is obtained at culture day 3.

11. The method of any of claims 1 to 4, comprising detecting a plurality of said biomarkers.

12. The method of any of claims 1 to 4, wherein said detecting at least one biomarker is performed by mass spectrometry.

13. The method of claim 12, wherein said mass spectroscopy is laser desorption/ionization mass spectrometry.

14. The method of claim 13, wherein said laser desorption/ionization mass spectroscopy is matrix-assisted laser desorption/ionization (MALDI).

15. The method of claim 13, wherein said laser desorption/ionization mass spectroscopy is surface-enhanced laser desorption/ionization (SELDI).

16. The method of claim 13, wherein the laser desorption/ionization mass spectroscopy includes:
(a) providing a substrate comprising an adsorbent attached thereto;
(b) contacting a test sample with the adsorbent;
(c) desorbing and ionizing at least one captured biomarker from the substrate; and
(d) detecting the desorbed/ionized biomarkers with a mass spectrometer.

17. The method of claim 16, wherein said adsorbent is a cation exchange adsorbent.

18. The method of claim 16, wherein said adsorbent is an anion exchange adsorbent.

19. The method of claim 16, wherein said adsorbent is an antibody adsorbent.

20. The method of claim 16, wherein said adsorbent is a biospecific adsorbent.

21. The method of claim 16, wherein said adsorbent is a bioselective adsorbent.

22. The method of any of claims 1 to 4, wherein the said at least one biomarker is measured by NMR spectroscopy.

23. The method of any of claims 1 to 4, wherein the said at least one biomarker is measured by immunoassay.

24. The method of claim 23, wherein said immunoassay is an enzyme immunoassay.

25. A kit for aiding in a diagnosis of developmental potential in an IVF-derived human embryo comprising one or more container means comprising an adsorbent comprising at least one capture reagent attached thereto, wherein the capture reagent binds at least one biomarker; wherein said at least one biomarker is selected from the group consisting of biomarkers having a molecular weight of about 2454±5, 2648±5.3, 2665±5.3, 2684±5.4, 2778±5.6, 4093±8.2, 7820±76, 45873±92, 282390±565 and 435170±870 Daltons.

26. A kit for aiding in a diagnosis of developmental potential in an IVF-derived human embryo comprising one or more container means comprising an adsorbent comprising at least one capture reagent attached thereto, wherein the capture reagent binds at least one biomarker; wherein said at least one biomarker is selected from the group consisting of biomarkers having a molecular weight of about 2454±5, 2648±5.3, 2665±5.3, 2684±5.4, 2778±5.6 Daltons.

27. A kit for aiding in a diagnosis of developmental potential in an IVF-derived human embryo comprising one or more container means comprising an adsorbent comprising at least one capture reagent attached thereto, wherein the capture reagent binds at least one biomarker; wherein said at least one biomarker is selected from the group consisting of biomarkers having a molecular weight of about 7820±76, 45873±92, 282390±565 and 435170±870 Daltons.

28. The kit of any of claims 25-27, further comprising instructions for using the said at least one capture reagent to detect said at least one biomarker.

29. The kit of any of claims 25-27, further comprising components for establishing one or more control population values or ranges.

30. The kit of any of claims 25-27, further comprising one or more containers with biomarker samples to be used as standard(s) for calibration.

31. The kit of any of claims 25-27, further comprising instructions for detecting a plurality of said biomarkers.

32. The kit of any of claims 25-27, wherein the capture reagent is a SELDI probe.

33. The kit of any of claims 25-27, wherein the capture reagent is a MALDI probe.

34. The kit of any of claims 25-27, wherein the capture reagent is an antibody that specifically binds to a biomarker.

35. The kit of any of claims 25-27, additionally comprising a cation exchange chromatography adsorbent.

36. The kit of any of claims 25-27, additionally comprising an anion exchange chromatography adsorbent.

37. The kit of any of claims 25-27, additionally comprising a biospecific adsorbent.

38. The kit of any of claims 25-27, additionally comprising a bioselective adsorbent.

39. The kit of any of claims 25-27, wherein the container means comprises a solid support selected from the group consisting of a chip, a microtiter plate, a bead and a resin.
